(19) United States
(12) Patent Application Publication
(10) Pub. No.: US 2006/0217549 A1
(43) Pub. Date: Sep. 28, 2006

(54) Diamine purification method

(76) Inventors: Didier Letourneur, Lyon (FR); Philippe Marion, Vernaison (FR)

Correspondence Address: Jean Louis Seugnet, Rhodia Inc CN 7500, 259 Prospect Plains Road, Cranbury, NJ, 08512-7500, US

(21) Appl. No.: 10/560,761

(22) PCT Filed: Jun. 25, 2004
(86) PCT No.: PCT/FR04/01613

§ 371 Date: Apr. 19, 2006

(30) Foreign Application Priority Data
Jun. 27, 2003 (FR) 0307816

(51) Int. Cl. C07D 257/02

(52) U.S. Cl. 540/474

(57) ABSTRACT

Process for the purification of diamines The present invention relates to a process for the purification of diamines obtained by hydrogenation of dinitriles. It relates more particularly to the purification of hexamethylenediamine obtained from the hydrogenation of adiponitrile. The process of the invention consists in treating a portion of the hexamethylenediamine stream withdrawn from the distillation column and in reinjecting this treated stream into the said column.

Process for the purification of diamines The present invention relates to a process for the purification of diamines obtained by hydrogenation of dinitriles.

It relates more particularly to the purification of hexamethylenediamine obtained from the hydrogenation of adiponitrile.

Diamines are important chemical intermediates, in particular in the manufacture of polymers. Thus, hexamethylenediamine is an important monomer in the manufacture of polyamides, in particular for the production of poly(hexamethylene adipamide).

For the manufacture of such polyamides, it is important and necessary for the hexamethylenediamine to exhibit very high purity characteristics. This is because the presence of impurities causes difficulties in obtaining a high degree of polymerization and results in particular in polymers with a yellowish colour. Furthermore, significant difficulties are encountered in the use of these polymers, in particular in the preparation of yarns or fibres, such as, for example, a high degree of breakage on spinning and drawing. In addition, these impurities can result in irregularities in the structure of the polymer and the cross section of the yarns, producing unevennesses in dyeing which are very harmful to the quality of the textile surfaces.

These characteristics of dyeing evenness are essential in the production of textile surfaces for clothing or for surface coverings.

Consequently, many studies have been carried out over very many years in order to obtain hexamethylenediamine with the highest possible purity or at least comprising the minimum of compounds which bring about declines in characteristics indicated above, when these were able to be identified.

Thus, a first process proposed which makes it possible to purify hexamethylenediamine consists in distilling this diamine in the presence of a base. Such a process is disclosed, for example, in Patents U.S. Pat. No. 2,987,452, EP 497 333 and GB 1 238 351. However, there are disadvantages to this process, such as, for example:
the control of the solubility of the bases,
the incineration of the heavy compounds formed during the distillation,
the need to use special materials which are resistant to corrosive media in the construction of the plants.

Other processes have been provided, such as, for example, the distillation of hexamethylenediamine in the presence of water or of an inert compound (WO 98/34900, WO 98/34901, WO 98/34902).

One of the aims of the present invention is to provide a novel process for the purification of diamines and more particularly of hexamethylenediamine which makes it possible to obtain significant removal of the troublesome compounds and thus a diamine which can be used in particular in processes for the manufacture of polyamides, more particularly polyamides for the manufacture of yarns or fibres.

To this end, the invention provides a process for the purification of aliphatic diamines which consist in treating the diamine or the reaction medium resulting from the synthesis of the diamine with hydrogen or a gas comprising hydrogen in the presence of a catalyst comprising an element chosen from the group consisting of platinum, palladium, ruthenium, rhodium, iridium, nickel and cobalt.

In a preferred embodiment of the invention, the diamine to be purified is present in the reaction medium resulting from its synthesis. The synthetic processes most generally used employ a hydrogenation of dinitrile compounds, such as the hydrogenation of adiponitrile to hexamethylenediamine, in the presence of a hydrogenation catalyst, such as Raney metals, for example Raney nickel.

In this embodiment, the hydrogenation treatment of the invention is carried out directly on the reaction medium resulting from the synthesis stage, optionally after separation of the unconverted dinitrile.

In another embodiment, the hydrogenation treatment of the invention can be carried out with a diamine extracted from the reaction medium, for example by distillation.

Whatever the embodiment of the treatment, the catalytic system used is advantageously of the supported catalyst type. The catalytic element or elements mentioned above are deposited, precipitated or adsorbed on a generally inert support according to techniques known to a person skilled in the art. Any support conventionally used in the field of supported catalysis can be used in the present invention.

By way of indication, mention may be made, as examples of a support suitable for the invention, of charcoals, titanium, zirconium or magnesium oxides, alumina or the like.

The concentration by weight of catalytic element is generally between 0.1% and 16% with respect to the element/support catalytic system.

Likewise, the hydrogenation stage of the invention can be carried out according to the usual techniques. Preferably, the techniques used are either the use of a stationary or fluidized bed catalytic system or suspending the catalyst in the reaction medium and recovering the catalyst by filtration or separation by settling.

The conditions for the implementation of the hydrogenation reaction are those known to a person skilled in the art for hydrogenation reactions. By way of indication, the reaction temperature is between 50° C. and 200° C., the hydrogen pressure being between 0.1 MPa and 5 MPa. Furthermore, it is possible to use a gas comprising hydrogen or to add, to the reaction medium, a hydrogen-generating compound.

After hydrogenation, the diamine is advantageously extracted from the medium by distillation, according to the usual techniques, or the hydrogenation treatment of the invention can be carried out directly on the reaction medium.

In another preferred embodiment of the invention, the process for the purification of the diamines is carried out concomitantly with the stage of distillation of the diamines.

In this embodiment, the reaction medium resulting from the hydrogenation of the dinitriles to diamines is fed, after extraction of the unreacted dinitriles, to a stage of distillation of the diamines, generally in the lower part of a distillation column. In such a process, the pure diamine is recovered at the column top.

According to the invention, a portion of the liquid stream circulating in the distillation column is withdrawn and fed to a hydrogenation reactor. The hydrogenation stage in accordance with the invention is carried out in this reactor. The resulting stream is fed to the distillation column, advantageously to a region situated upstream or downstream of the withdrawal region described above.

The process of the invention applies more particularly to the purification of linear or branched aliphatic diamines which comprise from 2 to 10 carbon atoms.

Hexamethylenediamine and methylpentanediamine, which are obtained by hydrogenation of adiponitrile or of methylglutaronitrile, are the preferred diamines among these diamines.

The purification process of the invention makes it possible to remove impurities present in these compounds, such as, in particular, tetrahydroazepine (THA).

Other impurities can be present in the diamine, such as hexamethylene or iminocyanocyclopentane (ICCP), for example.

In order to assess the quality of the diamines and in particular of hexamethylenediamine, in particular for use as monomer in the production of polyamide, it is conventional to determine the polarographic index of this compound, referred to as POLI.

This polarographic index represents in particular the concentration of imine compounds in the medium. It is determined by polarography and is expressed in moles of imine functional group per tonne of sample to be quantitatively determined.

Other details and advantages of the invention will become more clearly apparent in the light of the examples given below by way of illustration. These examples have a purely indicative nature.

### EXAMPLE 1

Hexamethylenediamine obtained by hydrogenation of adiponitrile in the presence of Raney nickel was distilled.

The concentration of the main impurities is as follows:
% by weight of THA: 0.113%
POLI, mmol/t: 7900

The hexamethylenediamine is introduced into a stirred reactor comprising a palladium-on-charcoal catalyst comprising 0.5% of palladium. The catalyst is sold by Engelhard.

The hydrogen pressure is 18 bar.

After reacting at 120° C. for 120 minutes, the hexamethylenediamine recovered comprises 0.0043% of THA and exhibits a POLI of 4000.

### EXAMPLE 2

A similar test to Example 1 was carried out in treating a hexamethylenediamine comprising 0.1066% of THA and a POLI of 12 000.

After treating at 120° C. for 120 minutes, the THA content fell to 0.0072% and the POLI to 1900.

### EXAMPLE 3

A hexamethylehediamine comprising 0.0406% of THA and a POLI of 4671 was treated at a temperature of 135° C. for 60 minutes according to the procedure of Example 1.

The hexamethylenediamine obtained exhibits a THA concentration of 0.00036% and a POLI of 244.

1-10. (canceled)

11. A process for the purification of aliphatic diamines, comprising the step of
a) carrying out an hydrogenation treatment on the diamine in a medium in the presence of a catalyst comprising platinum, palladium, ruthenium, rhodium, iridium, nickel or cobalt, and
b) recovering of the purified diamine.

12. The process according to claim 11, wherein in step a) the catalyst of the hydrogenation is a supported catalyst, the support of the catalytic element being charcoals, titanium, zirconiumoxides, magnesium oxides, or alumina.

13. The process according to claim 11, wherein in step b), the diamine is extracted from the medium of step a) after having preformed a hydrogenation by distillation.

14. The process according to claim 12, wherein in step b), the diamine is extracted from the medium of step a) after having preformed a hydrogenation by distillation.

15. The process according to one claim 11, wherein in step a) the medium is a reaction medium resulting from the synthesis of the diamine.

16. The process according to claim 15, wherein the synthesis of the diamine is a hydrogenation of a dinitrile compound.

17. The process according to claim 16, wherein the dinitrile present in the resulting reaction medium is separated before performing step a).

18. The process according to claim 11, wherein in step a), the catalyst is in the form of a stationary or fluidized bed.

19. The process according to claim 11, wherein in step a), the catalyst is in the form of a catalyst suspension.

20. The process according to claim 11, wherein in step a), the medium comprising the diamine is fed to a distillation column and a portion of the liquid stream circulating in the distillation column is withdrawn from a withdrawal point situated along the distillation column and is subjected to the hydrogenation reaction in the presence of the catalyst, said stream withdrawn after hydrogenation being fed back to the column upstream or downstream of the withdrawal point.

21. The process according to one claim 20, wherein in step a) the medium is a reaction medium resulting from the synthesis of the diamine.

22. The process according to claim 21, wherein the synthesis of the diamine is a hydrogenation of a dinitrile compound.

23. The process according to claim 21, wherein the dinitrile present in the resulting reaction medium is separated before performing step a).

24. The process according to claim 21, wherein in step a), the catalyst is in the form of a stationary or fluidized bed.

25. The process according to claim 21, wherein in step a), the catalyst is in the form of a catalyst suspension.

26. The process according to claim 11, wherein the diamine is hexamethylenediamine or methylpentanediamine.

27. The process according to claim 21, wherein the diamine is hexamethylenediamine or methylpentanediamine.
